(19) United States
(12) Patent Application Publication
(10) Pub. No.: US 2006/0235346 A1
(43) Pub. Date: Oct. 19, 2006

(54) Therapeutic light treatment devices and methods

(76) Inventor: Marvin A. Prescott, Los Angeles, CA (US)

Correspondence Address: BIOTECHNOLOGY LAW GROUP;C/O PORTFOLIOIP, PO BOX 52050, MINNEAPOLIS, MN, 55402, US

(21) Appl. No.: 11/402,157

(22) Filed: Apr. 11, 2006

Related U.S. Application Data
(60) Provisional application No. 60/594,509, filed on Apr. 13, 2005.

(51) Int. Cl. A61F 5/00

(52) U.S. Cl. 602/2

(57) ABSTRACT

Embodiments include a light therapy device including an insole base having a top surface configured to be disposed adjacent a human patient's foot and having at least one light emitter which is configured to emit light away from the top surface of the insole base and which may be disposed or concentrated in areas of the insole base corresponding to predetermined anatomic zones of the human foot.

### CROSS REFERENCE TO RELATED APPLICATIONS

This application claims priority under 35 U.S.C. section 119(e) from U.S. Provisional Patent Application Ser. No. 60/594,509, titled METHOD AND APPARATUS FOR LASER THERAPEUTIC TREATMENT OF FOOT CONDITIONS, filed Apr. 13, 2005, by M. Prescott, which is incorporated by reference herein in its entirety.

### BACKGROUND

Diabetes is a large and growing problem in the United States and worldwide, costing an estimated $45 billion dollars to the U.S. health care system. Patients afflicted with diabetes often have elevated glucose and lipid levels due to inconsistent use of insulin, which can result in a damaged circulatory system and high cholesterol levels. Often, these conditions are accompanied by deteriorating circulation and sensation in the nerves of the foot. As a result, diabetics experience a high number of non-healing foot ulcers.

It is estimated that each year up to three million leg ulcers occur in patients in the U.S., including venous stasis ulcers, diabetic ulcers, ischemic leg ulcers, and pressure ulcers. The national cost of chronic wounds is estimated at $6 billion. Diabetic ulcers often progress to infections, osteomyelitis and gangrene, subsequently resulting in toe amputations, leg amputations, and, occasionally, death. In 1995, approximately 70,000 such amputations were performed at cost of $23,000 per toe and $40,000 per limb. Many of these patients progress to multiple toe amputations and contralateral limb amputations. In addition, the patients are also at a greatly increased risk of heart disease and kidney failure from arteriosclerosis which attacks the entire circulatory system.

Some conventional methods of treatment for non-healing diabetic ulcers include wound dressings of various types, antibiotics, wound healing growth factors, skin grafting including tissue engineered grafts, and hyperbaric oxygen. In the case of ischemic ulcers, surgical revascularization procedures via autografts and allografts and surgical laser revascularization have been applied with short term success, but with disappointing long term success due to re-occlusion of the grafts. In the treatment of patients with venous stasis ulcers and severe venous disease, antibiotics and thrombolytic anticoagulant and anti-aggregation drugs are often indicated. The failure of these ulcers to heal and their frequent recurrence indicates a lack of success of these conventional methods. In addition, the number of pressure ulcers (i.e., bed sores) continues to grow with the aging of the population, and these can be particularly difficult to heal in bedridden or inactive patients. Accordingly, the medical community has a critical need for a low cost, portable, noninvasive method of treating diabetic, venous, ischemic, pressure ulcers and the like to reduce mortality and morbidity and reduce the excessive costs to the health care system.

Some conventional low power laser devices for treating such conditions may include a hand held probe with a single laser beam source, or a large stationary table console with attached probe(s) powered by a conventional fixed power supply. A common light source is a laser diode which is commercially available in varying power and wavelength combinations. Large probes which contain multiple laser diodes affixed to a stand are also known. Such large, multi-beam devices are typically very expensive and require extensive involvement of medical personnel when treating a patient. A large probe containing multiple beam sources is typically affixed to a stand which has to be focused and controlled by a doctor or ancillary medical personnel.

In addition to the cost of such conventional devices and the treatment therewith, such a device requires a patient to travel to the location of the laser treatment device in order to obtain the laser therapy. Studies have shown that such treatment typically must be provided on a regular basis (e.g., every few hours or daily for up to thirty minutes at each application) in order to be effective and to produce optimum results. This requires numerous patient visits to the treatment facility and extended treatment times at each visit to produce the desired effect. As it is common for problems to arise which necessitate the patient missing a treatment visit to the treatment facility, or for patients to be inconsistent in the times at which they are available for appointments, the efficacy of the treatment regimen may be lowered or the length of the treatment and the number of patient visits increased.

What has been needed are systems and methods for low power delivery of therapeutic light energy for treatment of difficult-to-heal ulcers, wounds and the like that are economical, convenient and more efficient than was previously possible. What has also been needed are systems and methods that can provide convenient low power delivery of therapeutic light energy without frequent recharging of batteries or medical visits and that can deliver therapeutic laser energy in an efficient manner directed specifically to target sites on a patient's body without the cost of a custom made device.

### SUMMARY

Some embodiments of a light therapy device include an insole base having a top surface configured to be disposed adjacent a human patient's foot and having at least one light emitter which is configured to emit light away from the top surface of the insole base and towards at least one predetermined anatomic zone of the human foot.

Some embodiments of a method of treating a human foot of a patient include providing a light therapy device including an insole base having a top surface configured to be disposed adjacent a human patient's foot and having at least one light emitter which is configured to emit light away from the top surface of the insole base and towards at least one predetermined anatomic zone of the human foot. The top surface of the insole base is disposed adjacent the patient's foot and the at least one emitter of the light therapy device is activated and therapeutic laser energy is delivered to at least on predetermined anatomic zone of the patient's foot.

Some embodiments of a kit for treatment of a human foot include a selection of at least a first light therapy device and a second light therapy device for treatment of a human foot. Each light therapy device includes an insole base having a top surface configured to be disposed adjacent a human patient's foot and having at least one light emitter which is configured to emit light away from the top surface of the respective insole base and towards at least one predetermined anatomic zone of the human foot. The first light therapy device is configured to emit therapeutic laser energy towards a first anatomic zone and the second light therapy device configured to emit therapeutic laser energy towards a second anatomic zone different from the first anatomic zone.

Some embodiments of a light therapy device include a shock absorbing insole base configured for insertion into a patient's shoe. The insole base has a top surface configured to be disposed adjacent a human patient's foot and has at least one light emitter which is configured to emit light away from the top surface of the insole base and towards at least one predetermined anatomic zone of the human foot. An electronic control and power charge circuit is configured to supply a patient with a programmed laser therapy regimen to stimulate increased local circulation of the foot to promote healing of foot conditions. The electronic control circuit is electrically coupled to a battery and a light source of the at least one emitter.

These features of embodiments will become more apparent from the following detailed description when taken in conjunction with the accompanying exemplary drawings.

### BRIEF DESCRIPTION OF THE DRAWINGS

FIG. 1 is an elevational view in section of a light therapy device embodiment including an insole base having an emitter module or.circuit coupled to a controller and power supply.

FIG. 2 is a top plan view of the light therapy device of FIG. 1.

FIG. 3 is an enlarged sectional side view of a laser circuit of the light therapy device of FIG. 1.

FIG. 4 is a top plan view of an embodiment of an insole base of a light therapy device having therapeutic light energy delivered to emitters through optical conduits.

FIG. 5 is an elevational view in section of the insole base of the light therapy device of FIG. 1 integrated into a shoe.

FIG. 6 is a block diagram of an embodiment of a power supply and control circuit of a light therapy device.

FIG. 7 is a plan bottom view of a pair of human feet indicating anatomic zones corresponding to reflex points for the heart, kidneys, pancreas, spleen and liver.

FIG. 8 is a bottom plan view of an insole base of a light therapy device with emitters positioned on the insole base in anatomic zones corresponding to reflex points of the kidneys, pancreas and heart.

FIG. 9 is a bottom plan view of insole bases of light therapy devices showing the disposition of multiple emitters concentrated in anatomic zones corresponding to reflex points of the liver and the heart.

FIG. 10 is a bottom plan view of insole bases of light therapy devices showing the disposition of multiple emitters concentrated in anatomic zones corresponding to reflex points of the kidneys.

FIG. 11 is a bottom plan view of an insole base of a light therapy device showing the disposition of multiple emitters concentrated in an anatomic zone corresponding to the reflex points of the pancreas.

FIG. 12 is a bottom plan view of an insole base of a light therapy device showing the disposition of multiple emitters concentrated in an anatomic zone corresponding to the reflex points of the spleen.

FIG. 13 is a bottom plan view of insole bases of light therapy devices showing anatomic zones corresponding to quadrants of a pair of human feet.

### DETAILED DESCRIPTION

Studies have shown that low power therapeutic light energy, which may include laser light energy, may be effective in the treatment of various medical conditions. For some embodiments, therapeutic light energy may include light at about 1 mW to about 500 mW in varying wavelengths of about 400 nm to about 1,300 nm delivered in intensities of about 0.5 J/cm² to about 10 J/cm². Studies have shown that delivery of therapeutic light, such as low power laser therapy (LLLT), stimulates fibroblasts and other cells important in the wound healing process to release a number of growth factors in greater amounts than would be released without laser photostimulation, thus enhancing and accelerating the wound healing process. Increased proliferation of fibroblasts and keratinocytes has been reported in a number of studies as well as the release of cytokines from Langerhans cells and the release of growth factors from macrophages.

Embodiments discussed herein are directed to methods and devices for applying therapeutic light, which may include low power therapeutic light, in the treatment of certain medical conditions. Specifically, some embodiments are directed to methods and devices for LLLT using vertical cavity surface emitting lasers (VCSELs) or other suitable emitters of therapeutic light energy to enhance healing of difficult-to heal wounds by promoting increased circulation and increased tensile strength of the healed wound. Therapeutic light may be generated by lasers, light emitting diodes (LEDs) or any other suitable light source such as incandescent bulbs, such as halogen bulbs, chemilluminescent sources, gas vapor bulbs and the like. Some conditions that may be treated may include, but are not limited to, healing diabetic ulcers, venous stasis ulcers, and pressure ulcers. Such treatment may also prevent the onset or the recurrence of such wounds if used in a prophylactic capacity for patients at risk of such conditions. Some embodiments are also directed to a methods and devices for balancing blood chemistry, stimulating the immune system, and improving endocrine function in diabetic patients as a result of stimulation of anatomic zones corresponding to reflex points or large arteries of the foot. Some embodiments may also be used in the treatment of peripheral neuropathy or prevention of the onset of peripheral neuropathy. Additionally, specific VCSELs, LEDs or other emitters of therapeutic light may be positioned adjacent the pedal arteries to increase general foot and leg circulation and to treat the blood and the immune system. For some embodiments, emitters 21 may be positioned or concentrated adjacent the dorsalis pedis artery, the arcuate artery and the dorsal digital arteries. For such embodiments, it may be desirable to position the emitters 21 on the top surface of the foot adjacent these arteries for better penetration and conduction of therapeutic light energy to these arteries.

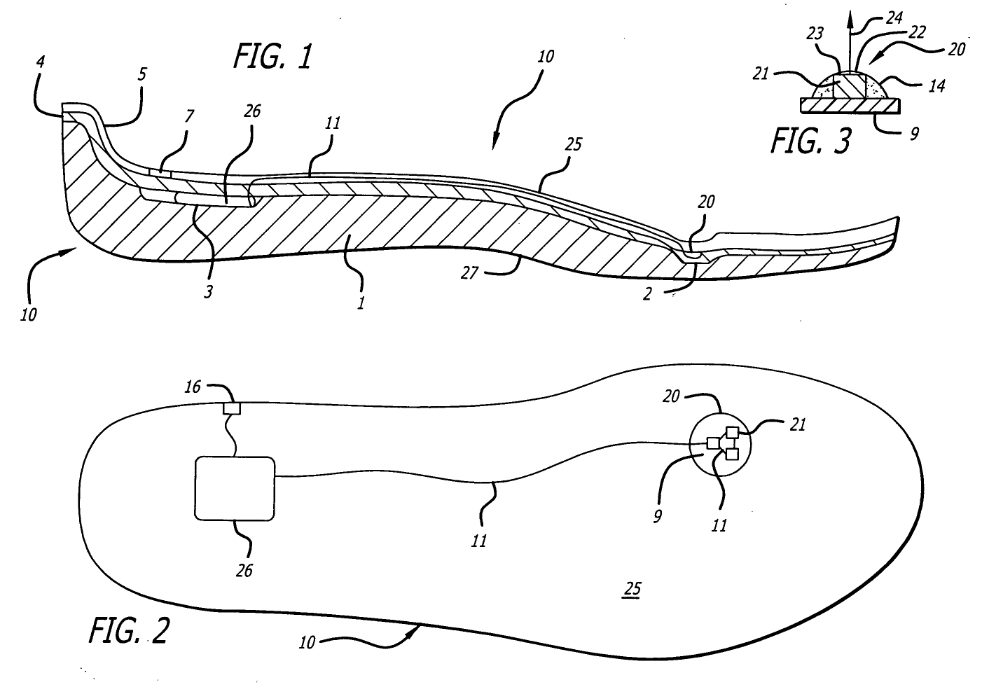

Referring to FIGS. 1-3, an embodiment of a laser or light therapy device 10 is illustrated. The light therapy device 10 includes an orthotic insole base 1 that may be made of polymer, composite fiber or other appropriate orthotic construction materials. A first relief area 2 is provided in the insole base 1 that corresponds to an anatomic zone or location of a wound, such as an ulcer, to be treated. A second relief area 3 is placed in the heel area of the insole base 1 in which the controller/power supply 26 is disposed. A cushioning layer 4, such as a porous material of approximately 1/10 inch thickness, is placed over the insole base 1 for cushioning and enclosing the controller/power supply 26.

A circuit 20 is disposed in the first relief area 2 of the insole 1 above the cushioning layer 4. Referring to FIG. 3, the circuit 20 includes an array of emitters or individual emitter 21 in the form of VCSELs 22 electrically connected in series, such as using conductive printed ink interconnects 11. The emitter 21 has an emission surface 23 that emits therapeutic light energy in a direction indicated generally by arrow 24 away from a top surface 25 of the insole base. The insole base also has a bottom surface 27. The plurality of emitters 21 are encapsulated in an optically clear epoxy material 14 to maintain the relative position of the emitters 21 and present a low profile for the circuit 20. The circuit 20 is further disposed on a substrate 9, which may be comprised of a flexible polyester material. The circuit 20 is operatively connected to the controller/power supply 26 via an electrical interconnect 11.

Although the emitters 21 shown in FIG. 1 include VCSELS 22, emitters 21 may have other configurations. For example, emitters 21 may include any suitable source or sources of therapeutic light, such as incandescent bulbs, such as halogen bulbs, chemilluminescent sources, gas vapor bulbs and the like. Emitters 21 may also include an output end of an optical conduit which is optically coupled to a light source or sources such as VCSELs 22, LEDs or any other suitable source of therapeutic light energy. For purposes the discussion herein, the terms “laser(s)” and “VCSEL(s)” may be used interchangeably. VCSELs 22 are semiconductor lasers which emit a beam normal to a surface of a semiconductor substrate. The semiconductor of the VCSEL may include aluminum arsenide (AlAs) or gallium arsenide (GaAs) or a combination thereof. Each VCSEL 22 may have a self-contained, high reflectivity mirror structure forming a cavity to produce the beam. Additional lenses may be used to focus or defocus an output beam thereof.

For some embodiments, the emitters 21 of the circuit 20 are activated by an activating switch 7. The activating switch 7 is a pressure switch which is operatively connected to the programmable controller/power supply 26. Switch 7 is activated by the patients'foot pressure or by a medical attendant. For such embodiments, when foot pressure is applied to the switch 7, the emitters 21 of the circuit 20 are activated, and, when foot pressure is released, the emitters 21 of the circuit are deactivated. For some embodiments of circuit 20, the emitters 21 of circuit 20 may be activated by an “on only” switch that is configured to activate emitters 21 of circuit 20 as soon as a battery (not shown) or other suitable electric energy storage device reaches a threshold charge level. Such an “on only” switch may be configured to deactivate the emitters 21 of the circuit 20 when the charge on the battery drops below a predetermined charge or storage level. The circuit 20 laser array and activating switch 7 are sandwiched between a hydrophobic biocompatible layer 5, such as a clear polymer layer of 0.5 millimeter thickness, and the cushioning material 4. The surface of the laser insole facing the foot surface follows the contour of the cushioning material layer, with the circuit 20 disposed in the relief area 2 to prevent any pressure on an ulcer adjacent the relief area. A recharge receptacle or recharge contact 16 is disposed on the side of the heel area on base 1, and is electrically connected to the controller/power supply 26.

Some embodiments of emitters 21 may have an output power of about 1.5 mW to about 15 mW, specifically, about 3.5 mW to about 12 mW, per emitter 21. Some emitter 21 embodiments may have an output wavelength of about 400 nm to about 1300 nm, specifically, about 650 nm to about 1300 nm, and, more specifically, about 700 nm to about 900 nm, and, even more specifically, about 760 nm to about 850 nm. Some VCSEL 22 embodiments may have dimensions of about 300 micrometers in length, about 200 micrometers in height and have an operational power threshold below about 12 mA and have a maximum output power of about 4 mW to about 5 mW at around 18 mA. Some VCSEL 22 embodiments may produce an output of therapeutic light of about 8 mW to about 12 mW at about 14 mA. As such, these VCSEL 22 embodiments consume very little power compared to conventional laser diodes and enable the use of multiple or numerous VCSELs 22 to be powered from a single battery or electrical energy storage source. Various forms of medical treatments using lasers and VCSELs 22 are disclosed in U.S. Pat. No. 5,616,140, issued Apr. 1, 1997, for METHOD AND APPARATUS FOR THERAPEUTIC LASER TREATMENT and U.S. Pat. No. 6,156,028, Ser. No. 09/025,874, filed Feb. 18, 1998, for METHOD AND APPARATUS FOR THERAPEUTIC TREATMENT OF WOUNDS, by M. Prescott, both of which are incorporated by reference herein in their entirety.

For some embodiments, a VCSEL chip with sub-mount for surface mounting (chip mounts) requires only about 150 microns in height including the sub-mount. The sub-mount may include a heat sink material such as silicon, ceramic copper, or aluminum nitride, and contacts (i.e., anode and cathode) are positioned so that the VCSEL can be surface mounted on a circuit, such as the circuit 20 of FIGS. 1-3. The VCSEL may be mounted on the sub-mount and wire-bonded to the sub-mount or alternatively flip-chip bonded. In the flip-chip version, both contacts would be on the bottom of the unit, thus increasing the manufacturing reliability. The VCSEL chip may be encased in an optically clear epoxy encapsulant, resulting in a low-profile laser device. A single VCSEL 22 may be contained in chip embodiments or an array of VCSEL emitters 21 may be used in chip embodiments, each chip having about 2 to about 4 VCSELs 22. A number of emitters 21 having different wavelengths could be combined with each chip having its own specific wavelength, with those wavelengths ranging from about 400 nm to about 1300 nm. The VCSEL 22 devices may then distributed on the circuit material in accordance with the design of the device and are interconnected using electrical connectors or by printed conductive ink interconnects. A battery (not shown), which may be a polymer battery, may be surface mounted on the reverse side of the circuit carrying the controller/power supply 26 or attached to the controller/power supply and electrically coupled to the controller/power supply 26. The battery may also be covered with a clear, biocompatible polymer which may have a thickness of about 0.5 mm and which may be scaled to the cushioning layer of the orthotic insole.

The programmable controller/power supply 26 provides power and timing control for operation of the emitters 21. The programmable controller/power supply 26 may be initiated by a single-pole, double-throw switch, or by embodiments of pressure switch 7 as discussed above. The timing control performed by the controller/power supply 26 includes initiating the operation of the emitters 21 for a predetermined time period in accordance with a prescribed or predetermined therapeutic light treatment regimen. A control device embodiment performing such a function may include a programmable controller having a 24-hour timing function which initiates operation of the emitters 21 for a predetermined period of time over the course of a 24-hour period. Embodiments of the therapeutic device 10 may be programmed to deliver two minutes of laser therapy at four-hour intervals for about 48 hours at which time the battery would be recharged or a new battery installed. To prevent the controller/power supply 26 of the light therapy device 10 from being accidentally deprogrammed during a critical healing period, the switch 7, may be an “on-only” switch that cannot be turned off by the patient as discussed above.

For some embodiments, when the patient inserts the light therapy device 10 inside an article of flexible footwear such as a shoe, sandal, slipper, sock or the like, and stands erect, the pressure switch automatically initiates a preprogrammed treatment regimen. After removal of foot pressure, the pressure switch 7 would open and the patient would be required to manually trip the pressure switch and apply the insole base to the foot surface by replacing the light therapy device 10 inside the article of footwear. For some embodiments, the light therapy device 10 may include a standard on/off switch that does not initiate programming of the light therapy device 10, but rather initiates emitter 21 activation immediately. For some embodiments, the switch may include an “on only” switch as discussed above.

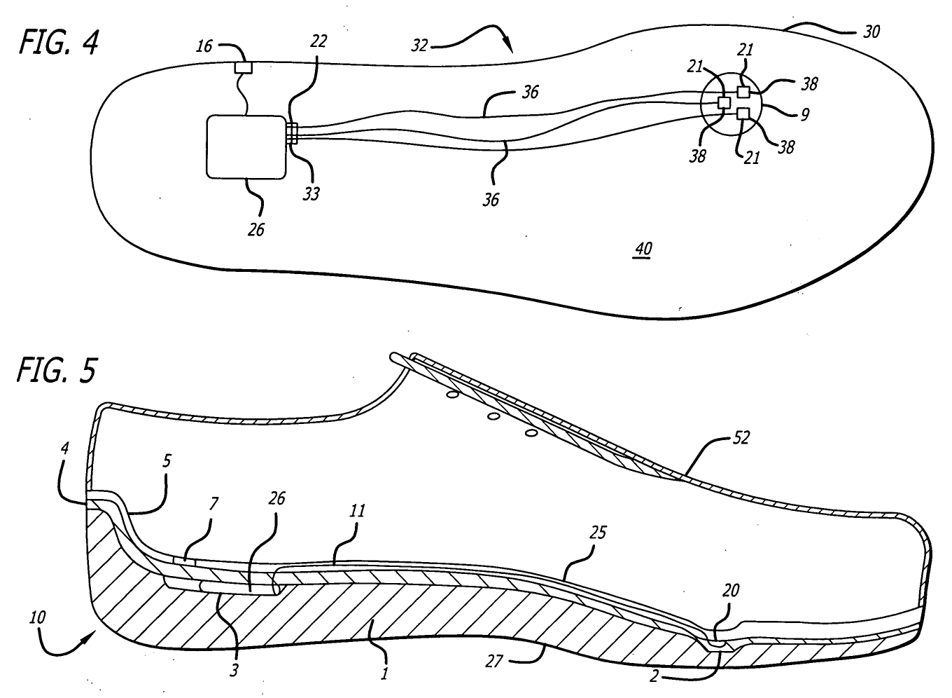

FIG. 4 is a top plan view of an embodiment of an insole base 30 of a light therapy device 32 having therapeutic energy delivered to emitters 21 through optical conduits 36. The insole base 30 may have the same or similar features, dimensions and materials as those of the insole base 1 discussed above. However, in this embodiment, therapeutic light energy is generated by a light source 33 in the form of a VCSEL or VCSELs 22 which may be disposed adjacent the controller/power supply circuit 26 a shown in FIG. 4. Once generated, the therapeutic light is then carried from the light source 33 through the optical conduits 36 to an output end 38 of the optical conduit and then emitted from corresponding emitters 21. Emitters 21 may be the output end of the optical conduit 36 which is curved or redirected in order to direct the therapeutic light generally away from a top surface 40 of the insole base 30. Emitters 21 may also include a reflective surface (not shown), such as a mirror or the like, to redirect the output of the optical conduit or conduits 36. The optical conduits 36 may include standard multi-mode or step index optical fibers, such as plastic clad fibers and the like, or any other suitable optical conduit. The configuration of the light therapy device 32 allows for a single light source 33 to be disposed adjacent the controller/power supply circuit, or anywhere else on the insole base 30, and have the emitters 21 disposed in any desired location or locations. Such an embodiment may be useful for lowering the cost of manufacturing of the light therapy device 32. Although VCSEL 22 is shown as the source of therapeutic light, any suitable source may be used such as any of the sources discussed above.

In operation, the light therapy devices 10 and 32 may be used to accelerate and enhance healing of a foot ulcer or wound by promoting angiogenesis, increased circulation, and increased tensile strength of the wound by increasing collagen deposition in the wound. In the case of a bone fracture, light therapy devices 10 and 32 may be used to accelerate the healing of the bone in the foot area. Thus, in operation, embodiments of the light therapy devices 10 and 32 may be placed inside the patient's shoe, slipper, sandal or the like by the physician or ancillary medical personnel or worn inside a sock to deliver a programmed laser biostimulation treatment regimen. An appropriate clear wound dressing would be placed first to minimize attenuation of the therapeutic light. The emitters 21 may be positioned in the relief area 2 of a insole base 1 and focused on the area of an ulcer. In the case of a pressure ulcer on a patient's heel, a strip of emitters 21 may be placed in the heel area of the insole 1 posterior to the controller/power supply 26. Alternatively, the emitters 21 may be distributed over the entire surface of the orthotic insole 1 facing the foot bottom in an off-the-shelf version of light therapy device 10.

FIG. 5 is an elevational view in section of an embodiment of a light therapy device 50 wherein the insole base 1 of the light therapy device 10 of FIGS. 1-3 has been integrated into a standard footwear embodiment, such as the shoe 52 shown. Although the insole bases discussed herein are directed generally to thin, flexible embodiments that would allow insertion of the insole bases into an article of footwear such as a patient's shoe, slipper, sandal, sock or the like without modification of such footwear, it may also be desirable to have the features of the insole base 1, 30 or others discussed herein, incorporated into a shoe, slipper, sandal, sock or the like for the convenience of use of a patient. Such an embodiment 50 may also provide more available volume for battery and circuit components for embodiments that include a large number of emitters or light sources, a longer period of use without recharging the battery, or both.

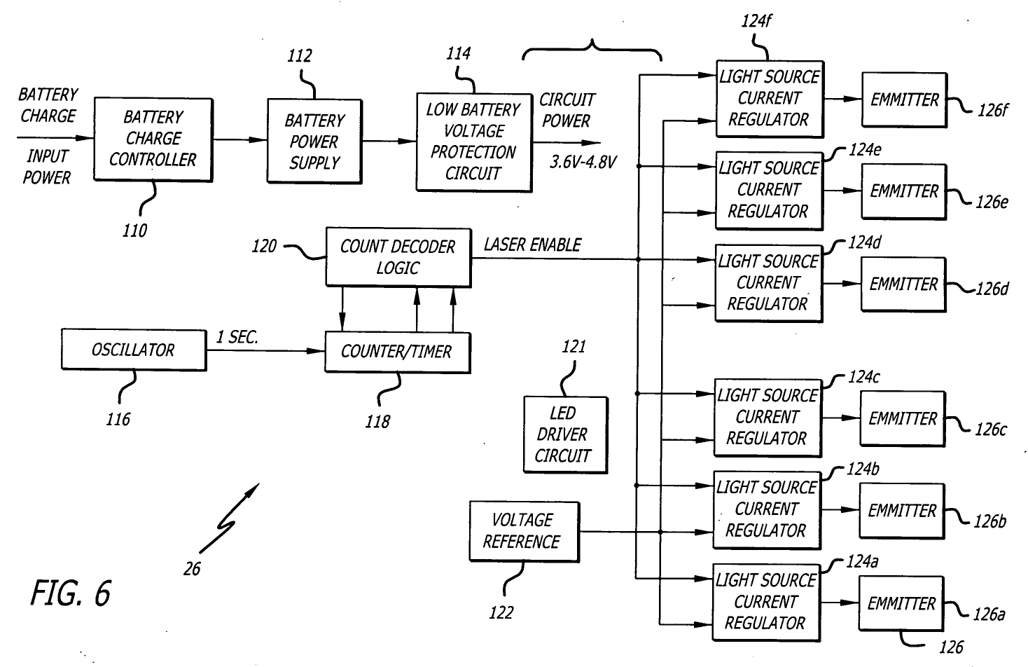

An embodiment of a controller/power supply circuit 26, which may be used with any of the light therapy device embodiments discussed herein, is shown in FIG. 6. A battery charge controller 110, which may be connected to an external power source, supplies a battery power supply 112 with a charge when the charge controller 110 is connected to the external source. When an optimum charge level is reached, the charge controller ceases supplying the battery 112 with the charge. In some embodiments, the battery 112 is capable of maintaining a charge sufficient for one week of laser therapy based on a treatment being provided for two minutes every four hours or a duty cycle of less than 5% however, a different duty cycle may be selected based on the application. Some embodiments may require recharging after about 48 hours of use. A low battery voltage protection circuit 114 regulates the power supplied by the battery 112 and provides a voltage output between 3.6 and 4.8 volts. The protection circuit 114 ceases the supply of power if the voltage drops below the threshold level of 3.6 volts to avoid damage to the circuit components. The power supplied by the protection circuit 114 is used to power the circuit components as well as the emitters 21 or other light sources of a light therapy device 10 or 32. An oscillator 116 is provided which supplies pulses at one second intervals to counter/timer circuit 118. The counter/timer circuit 118 counts the pulses while a count decode logic circuit 120 monitors the count.

The count decode logic circuit 120 is a multipurpose logic circuit which may include, for some embodiments, a PAL (programmable array logic) or a PLA (programmable logic array) that may be programmed to detect certain counts, e.g., 14,400 which would correspond to four hours of time and 120 which would correspond to two minutes of time. The count decode logic circuit 120 may be capable of maintaining the stored timing program (and, therefore, the prescribed regimen) without power being applied thereto. The count decode logic circuit 120 may also include a discrete logic, circuit formed of standard logic components. While such a circuit may be more cost effective from a low-volume manufacturing perspective, some count decode logic 120 embodiments include a programmable logic circuit to afford maximum flexibility in operation of a light therapy device 10 or 32.

Upon detection of the programmed count, the decode logic circuit 120 outputs a light emitter enable pulse which enables light source current regulator circuits 124a-124f which regulate the power to each light emitter 126a-126f (corresponding to the emitters 21 of FIGS. 1-3). The regulator circuits 124a-124f, which may compare the current with a known voltage reference in order to maintain a constant current output, receive a voltage reference input from a voltage reference circuit 122, the voltage reference circuit 122 may include an active bandgap Zener diode which supplies a constant voltage output (e.g., on the order of 1.2 to 1.5 volts) regardless of the voltage of the battery 112. At the same time, the count decode logic 120 provides a RESET pulse to the counter/timer circuit 118 to reset the count, and the counter/timer circuit 118 continues counting the pulses from the oscillator 116.

The light emitter enable pulse remains active for the programmed length of treatment, e.g., two minutes, or 120 counts of the counter/timer circuit 118. While enabled, the current regulators 124a-124f use the input from the voltage reference circuit 122 to provide a predetermined amount of current to produce therapeutic light having a desired power level, such as about 3.5 mW to about 10 mW for some embodiments. The therapeutic light is produced by the emitters 126a-126f. The logic circuit 120 continues to monitor the count in the counter 118 and detects when the count reaches a programmed amount corresponding to the prescribed treatment length (e.g., 120 counts) and then terminates the light emitter enable pulse. At the same time, the logic circuit 120 provides a RESET pulse to reset the count in the counter/timer circuit 118, and the cycle begins again.

To preserve battery power, the count decode logic circuit 120 may be programmed to provide a pulse to individual ones of the regulator circuits 124a-124f. This configuration permits sequential firing of individual emitters 21 of the emitter 21 arrays rather than simultaneous firing of all emitters 21 of an emitter array simultaneously. Thus, particular areas of the wound or ulcer area may be pinpointed for therapeutic light treatment. Alternatively, multiple therapeutic light enable pulses may be provided.

For some embodiments, the controller/power supply circuit 26 may be disposed on a single circuit board which may be sufficiently thin (e.g., on the order of less than 1 mm) to be encapsulated by a polymer sheet and be formed integral therewith. For some embodiments, the controller/power supply circuit 26 may also include multiple circuit components which are readily available from electronics suppliers or may be implemented in an application specific integrated circuit (ASIC) to reduce size and complexity thereof. Referring again to FIG. 2, the circuit 20 may be from on a non-conductive polyester material in which the electrical interconnects and circuit design are printed with flexible, electrically conductive ink, such as developed by Polyflex Circuits Corporation. Flexible circuits may also be made using ULTEM (a trademark of General Electric Corp) or Kapton (a trademark of Dupont Corp). The emitters 21 may be sealed by a clear epoxy chip encapsulant 14 shown in FIG. 3 and the circuit 20, controller/power supply circuit 26 and pressure switch 7 are fixed and sealed to the cushioning 4 layer with a biocompatible clear hydrophobic polymer layer of about 0.5 mm thickness, which results in a smooth surface on the top side of the insole base 1 facing a bottom of a patient's foot.

Some embodiments include a flexible printed circuit and interconnects, printed on flexible battery material which may be fashioned to the shape of a plantar surface of the foot or other appropriate body area shape and may have a custom designed system on a chip (SOC) which controls the sequential activation or firing of the emitters 21 in the form of VCSELs 22 or other light sources which may be arranged in the already described predetermined pattern or patterns for the foot or alternate patterns for other body areas. The thin, flexible battery/circuit containing the emitters 21 are housed between two thin layers of flexible polymer with the polymer over the emitters 21 being optically clear. These embodiments may be thin enough and flexible enough to be attached to the bottom of the foot by a medical adhesive in a similar fashion to a self adhering bandage to provide a therapeutic treatment regimen to patients who are non ambulatory or bed ridden. In addition, such embodiments may take various shapes and sizes to conform to most parts of the body for example ankles, legs, arms, torso, knee joints, and other joints and would be affixed to the area of interest or injury by a medical adhesive. Some embodiments may be in the form of sheets of battery material upon which the circuits and interconnects may be printed and diodes may be mounted. Such embodiments may be installed in cushions, seat pads, wheel chair pads, bed pads, or the like in order to stimulate circulation and for example prevent and treat decubitus ulcers in bed ridden or wheel chair bound persons.

The controller/power supply circuit 26 may include a 6 volt, wafer thin, flexible polymer battery by ECR Ltd., Israel, and a programmable controller. The ECR battery technology includes hydrogen ion storage electrodes and an extremely high rate solid state electrolyte, is rechargeable and completely environmentally friendly. The technology allows manufacture as conformable films. ECR battery embodiments may also be printed directly on flexible circuit material and be capable of one minute quick recharge without damaging the battery which would allow duty cycles greater than 5% for some light therapy device 10 embodiments. The battery may also include a simple 3-6 Volt battery or a rechargeable nickel-metal hydride battery. It may be desirable, in some embodiments, for the battery to provide sufficient power for about 2 days to about 7 days of a treatment regimen. For some embodiments, a transformer or other appropriate power supply may be used that would transform household AC voltages to DC voltages for use by the light therapy device 10.

As discussed above, the operation of the therapeutic device 10 may be initiated by switch 7. The switch 7 may have an LED or other visual or audio indicator incorporated therein to indicate function or battery status of the device 10. Embodiments of the switch 7, which may also be covered by the biocompatible polymer layer 5, is a pressure switch that activates the preprogrammed treatment regimen but automatically disengages and shuts off the system when no pressure is applied for a predetermined time period, such as 30 minutes. This allows therapeutic light therapy to be applied while the patient is wearing the device and saves battery power when the patient is not wearing the device. Alternatively, an on/off switch would activate the device if it is to be worn inside a sock, slipper or shoe, or may be directly affixed to the foot when the patient is sleeping or is non-ambulatory. If an on/off switch version is selected, a time period can be provided between the operation of the switch 7 and the actual initiation of the light therapy treatment regimen to allow sufficient time for the therapeutic device 10 to be properly positioned on the patient's foot prior to initiation of therapeutic light therapy.

In the case of a diabetic ulcer, a clear hydrogel dressing (e.g., Intrasite by Smith & Nephew) may be applied and then a clear polyurethane hydrocellular dressing (e.g., OpSite by Smith-Nephew or Omiderm by ITG), may be placed over the hydrogel to prevent bacterial contamination of a wound. The polyurethane protective film may help prevent bacterial contamination of the light therapy device 10 and allows penetration of the therapeutic light in the treatment area without significant attenuation of the beam of therapeutic light emitted from the light therapy device 10. In some embodiments, a polyurethane hydrocellular dressing alone such as an OpSite or Omiderm dressing may be placed over the wound. During treatment, this type of dressing prescription would allow once a week change of the dressing and increase the efficiency of healing.

After the patient or medical personnel places the light therapy device 10 in the shoe or shoes and the patient puts on the shoes, foot pressure on the pressure switch 7 activates the system and emission of therapeutic light energy begins. In operation, the therapeutic laser energy from the light therapy device 10 irradiates the appropriate treatment area of the foot ulcer. Specifically, the emitter arrays 21 are repetitively fired at the appropriate wavelength and power so as to penetrate the patients foot and interact with the tissue thereof. Therapeutic light energy having wavelengths of about 400 nm to about 1300 nm may be selected, although some embodiments utilize a wavelength of about 780 nm or about 850 nm.

Therapeutic devices 10 having a different treatment regimen preprogrammed therein may be provided, with a physician selecting a particular device in accordance with an appropriate regimen depending on the patients'condition. Alternatively, the controller/power supply 26 may be provided with a PCMCIA port which interfaces with a so-called “smart card” or master programming card which can be inserted therein and a treatment regimen may be downloaded to the controller 30 by the treating physician.

After being placed in the patient's footwear, e.g., shoe, slipper, sandal, sock or the like, the patient simply wears the light therapy device 10 for the prescribed time period. The light therapy device 10 automatically delivers the prescribed therapeutic laser light therapy as determined by the programmable controller/power supply 26. Thus, an efficient, programmed laser treatment regimen over a prescribed time period may be conveniently delivered. In the treatment of general foot problems, the laser insole device 10 could be stocked in an off-the-shelf adaptable version to be used for a variety of foot injuries and fractures in a routine or an emergency basis. In these embodiments, a number of emitters 21 may be distributed over the top surface of the light therapy device 10 or 32.

Such treatment methods using the light therapy device 10, 32 or any other embodiment discussed herein provide freedom and convenience to the patient. For example, depending on the nature of the prescribed therapeutic light therapy, the patient may only need to wear the therapeutic device 10 during certain hours of the day (e.g., while sleeping) or full time, without interfering with a normal lifestyle. The device can be easily and rapidly recharged to provide extended treatment times. Additionally, the patient's visits to the physician can be reduced to a minimum and the patient can wear the device on a long term basis to maintain the improvement in circulation and tissue health, thus reducing the potential for further ulceration, infections, and life threatening amputations.

Some embodiments are directed to a method and apparatus for light therapy treatment of foot conditions such as ulcers, wounds, nerve injuries, tendon and ligament injuries, joint injuries, fractures, peripheral vascular disease, peripheral neuropathy and circulation deficits via photon stimulation of local circulation, general circulation and specific foot acupuncture or reflex points. In addition, it may be desirable to provide a device that can be used to treat foot conditions in any area of a patient's foot with an “off the shelf” light therapy device or kit of light therapy devices. Some embodiments allow simplification in the manufacturing process by having a predetermined pattern of emitters 21 which provide treatment to many likely locations on the foot. Such embodiments avoid the need for customization of a light therapy device to a specific patient and, as such, may provide immediate treatment when a delay in starting treatment could result in a worsening of the patient's condition. Various sizes of insole bases may also be made available including S, M, LG, XL, saving costly medical personnel time to fit the light therapy device.

For some embodiments of therapeutic light devices, certain emitters 21 are positioned to stimulate local circulation on the bottom of the foot while other emitters 21 may be positioned to direct therapeutic light to the large foot arteries such as the dorsalis pedis artery, the arcuate artery and the dorsal digital arteries to stimulate the general circulation of the foot. As discussed above, some embodiments also provide specific locations of emitters 21 to stimulate foot acupuncture points of the pancreas, spleen, heart, and kidney. Such stimulation may aid in the treatment of diabetic ulcers as well as other conditions.

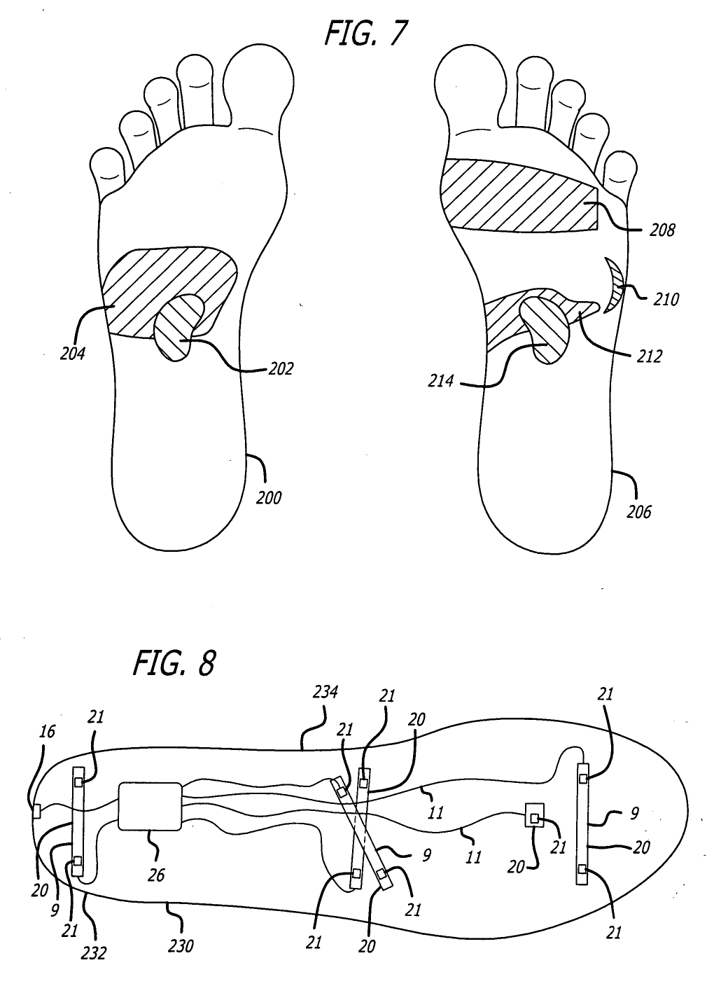

FIG. 7 is a plan bottom view of a pair of human feet indicating anatomic zones corresponding to reflex points for the heart, kidneys, pancreas, spleen and liver. Stimulation of the tissue of a patient's foot in these anatomic zones may produce a result similar to that of mechanical acupuncture in theses zones. As such, improved functioning of a patient's heart, liver, spleen, pancreas and kidneys, as well as other organs, may be achieved by selective optical stimulation of these anatomic zones. The right foot 200 includes an anatomic zone 202, indicated by the bounded cross hatched area, corresponding to the kidneys and an anatomic zone 204 corresponding to the liver. The left foot 206 includes an anatomic zone 208 corresponding to the heart, anatomic zone 210 corresponding to the spleen, anatomic zone 212 corresponding to the pancreas and anatomic zone 214 corresponding to the kidneys.

FIG. 8 illustrates a bottom plan view of a laser insole base 230 of a light therapy device 231 having a predetermined pattern of emitters 21 strategically embedded in the front, middle and heel areas of the insole base 230. Insole base 230 may have the same or similar features, dimensions and materials as those of insole base 1 discussed above. Such a configuration may deliver photon therapy or therapeutic light to desirable or predetermined anatomic zones without the need to customize the device for each patient. Such predetermined pattern of emitters 21 allows manufacturing of an off the shelf device in a range of sizes which medical personnel can place in the patients shoe at the initial visit to promote healing of wounds and injuries. Some emitters 21 may be arranged to treat local circulation of the foot and other emitters 21 may be positioned to focus on the plantar arteries to improve the general leg circulation. Certain other emitters 21 are positioned to stimulate specific foot acupuncture or reflex points of the pancreas, spleen, kidney and heart-major organs which are affected by diabetes. Alternatively, the device may be adapted as a flexible bandage that can be applied to the skin. Insole base 230 may have the same or similar features, dimensions and materials as those of the insole bases 1 and 30 discussed above.

Relief areas such as relief area 2 and relief area 3 as shown in FIG. 1 may be provided in the upper surface of the insole base 230 which allows VCSELs 22 or other light sources of the emitters 21 to be mounted on heat conducting mounting strips 9 to be positioned in predetermined patterns as shown in FIG. 8. A similar relief area may be placed between the heel area 232 and the midsole area 234 of the insole base 230 in which the controller/power supply 26 is disposed. A cushioning layer 4 of shock absorbing, sheer reducing, conformable polymer such as Plastazote® may placed over the insole base 230 for cushioning and enclosing the controller/power supply circuit 26 and diode modules 20 and interconnects 11.

The laser diode circuits 20 may be disposed in the relief area above the base area of the insole base 230. The circuits 20 include individual emitters 21 in the form of VCSELs 22 arranged in a specific pattern mounted on heat conducting strips 9 and connected in series on the heat conducting strips 9. A cutout in the top cushioning layer at the location of each emitter 21 is large enough to allow unobstructed transmission of the emission of therapeutic light from a top surface (not shown) of the insole base 230. The emitters 21 may be encapsulated in an optically clear epoxy material 14 to protect them from moisture and debris as shown in FIG. 1. The circuits 20 may be operatively connected to the controller/power supply 26 by an electrical interconnects 11.

The emitters 21 of the circuit 20 may be activated by an “on-only contact switch 7 which is operatively connected to the programmable controller/power supply 26. The therapeutic device may also be activated by an on/off switch or a pressure switch. To prevent the therapeutic device not being activated by the patient or from being accidentally deprogrammed during the critical healing period, it may be desirable for switch 7 to be an “on-only” switch that once activated by the treating medical personnel, cannot be turned off by the patient. A recharge receptacle 16 may be disposed at the heel area on base 1 and is electrically connected to the controller/power supply 26.

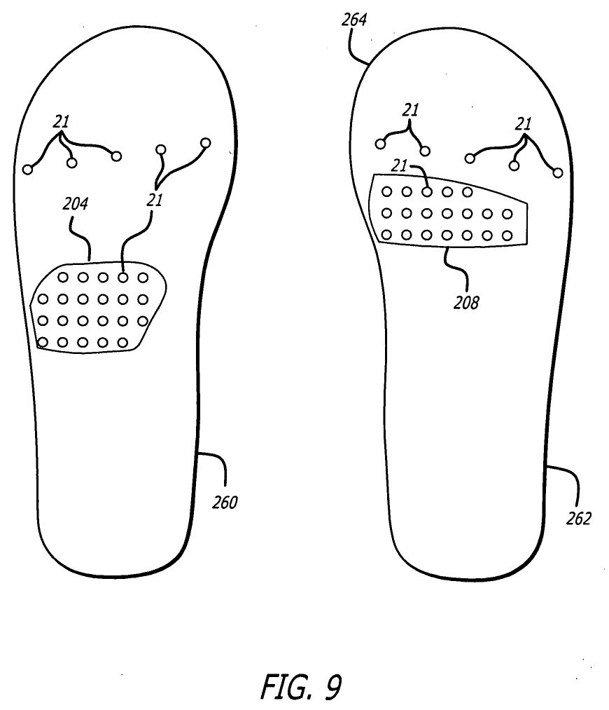

FIG. 9 is a bottom plan view of insole bases of light therapy devices showing the disposition of multiple emitters 21 concentrated in anatomic zones corresponding to reflex points of the liver and the heart. A bottom view of a right foot insole base outline 260 shows a plurality or array of emitters 21 disposed substantially within the anatomic zone 204 of the right foot outline that corresponds to a zone of reflex points for the liver. The anatomic zone 204 is substantially rectangular and disposed in the mid-section of the right foot outline 260 extending substantially across the width of the foot outline and along a front to back direction over a distance of about one quarter the total length of the foot. A bottom view of a left foot insole base outline 262 shows a plurality or array of emitters 21 disposed substantially within an anatomic zone 208 of the left foot outline that corresponds to a zone of reflex points for the heart. The anatomic zone 208 has a substantially rectangular shape extending from an inside edge 264 of the left foot outline 262 across approximately two thirds to three quarters the width of the foot outline. The size of the anatomic zone is similar to that of the anatomic zone 204.

FIG. 9 also shows a set of 5 emitters 21 spaced across the front portion of the right insole base outline 260 and a set of 5 emitters 21 spaced across the front portion of the left insole base outline 262. These arrays of emitters 21 are disposed at approximately the location on the insole outlines corresponding to the base of the toes of a patient's foot. Each emitter 21 is also configured to be disposed adjacent a corresponding dorsal digital artery of the patient's foot. By activating emitters 21 disposed in such anatomic zones corresponding to the major pedal arteries such as the dorsal digital arteries, circulation in the dorsal digital arteries may be improved. In addition, the combined effect of applying therapeutic light to the dorsal digital arteries, or other pedal arteries, such as the dorsalis pedis artery and the arcuate artery may improve circulation in the larger upstream arteries such as the anterior tibial artery and posterior tibial artery of the patient.

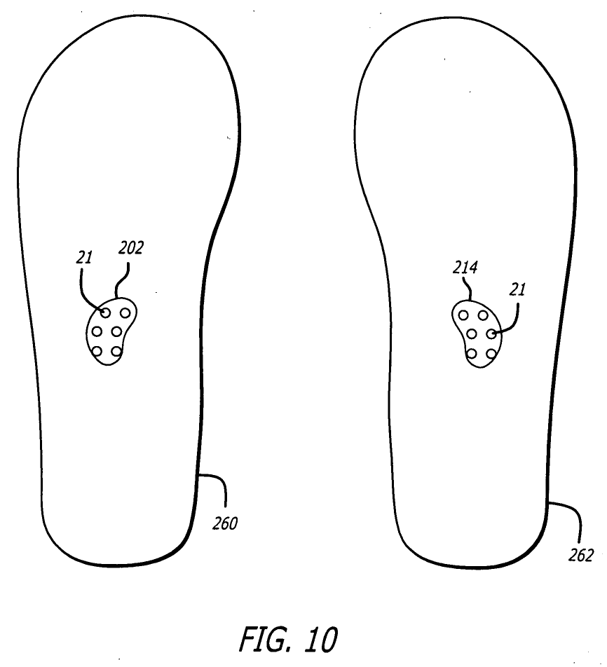

FIG. 10 is a bottom plan view of insole bases of light therapy devices showing the disposition of multiple emitters concentrated in anatomic zones corresponding to reflex points of the kidneys. A bottom view of a right foot insole base outline 260 shows a plurality or array of emitters 21 disposed substantially within an anatomic zone 202 of the right foot outline that corresponds to a zone of reflex points for the kidneys. The anatomic zone 202 is an oval shaped zone substantially centered in the foot outline having a longitudinal dimension or major axis extending front to back over a distance of about one fifth to about one seventh the overall length of the foot outline. A minor axis or transverse dimension of the anatomic zone is about one fourth the width of the foot outline at the midsection of the foot outline. A bottom view of a left foot insole base outline 262 shows a plurality or array of emitters 21 disposed substantially within an anatomic zone 214 of the left foot outline that corresponds to a zone of reflex points for the kidneys. The anatomic zone 214 has substantially the same size, shape and relative position with respect to the left foot outline as anatomic zone 202 with respect to the right foot outline.

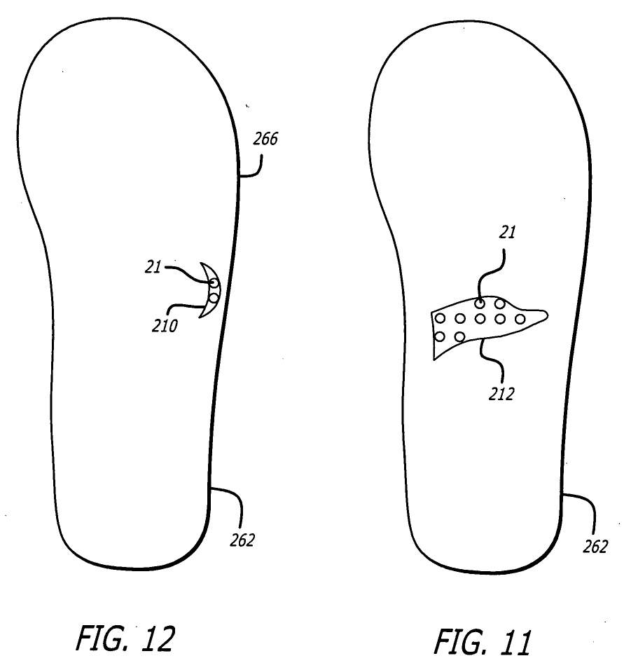

FIG. 11 is a bottom plan view of an insole base of a light therapy device showing the disposition of multiple emitters 21 concentrated in the anatomic zone 212 corresponding to the reflex points of the pancreas. A bottom view of the left foot insole base outline 262 shows a plurality or array of emitters 21 disposed substantially within the anatomic zone 212 of the left foot outline 262 that corresponds to a zone of reflex points for the pancreas. The anatomic zone 212 is a somewhat cone-shaped or sock shaped zone disposed over the front to back center of the foot outline. The anatomic zone 212 extends from the inside edge 264 of the left foot outline across a distance of about two thirds to three quarters the width of the left foot outline at the midsection position. The width of the anatomic zone 212 in a front to back direction is about one sixth to about one eighth the front to back length of the foot outline 262.

FIG. 12 is a bottom plan view of an insole base of a light therapy device showing the disposition of multiple emitters concentrated in an anatomic zone corresponding to the reflex points of the spleen. A bottom view of the left foot insole base outline 262 shows a plurality or array of emitters 21 disposed substantially within the anatomic zone 210 of the left foot outline 262 that corresponds to a zone of reflex points for the spleen. The anatomic zone 210 is a tear dropped shaped zone slightly forward of the front to back center of the foot outline and disposed towards an outside edge 266 of the left foot outline 262. The anatomic zone 212 has a size about one half that of the anatomic zones 202 and 214 with a longitudinal dimension or major axis extending in a front to back orientation with respect to the foot outline. The spacing between multiple emitters 21 concentrated in the anatomic zones discussed herein may be about 0.5 cm to about 2.0 cm for some embodiments although other spacings between multiple emitters 21 in contemplated. Other spacings may depend on the type of emitter 21. In addition, the patterning of multiple emitters 21 concentrated in an anatomic zone or zones may vary from embodiment to embodiment. The patterning of emitters 21 in FIGS. 9-12 is shown as a regularly spaced orthogonal grid pattern, however, other patterns such as spiral, concentric rings, spoked or any other suitable pattern that delivers a desired therapeutic light intensity to an anatomic zone or sub-zone within an anatomic zone may be used. Although the emitters 21 shown in FIGS. 9-12 do not show details of supporting structure, such as relief areas 2, circuits 20, controller/power supplies 26 or the like, the same or similar structures may be used to mount emitters 21 in the patterns shown in FIGS. 9-12.

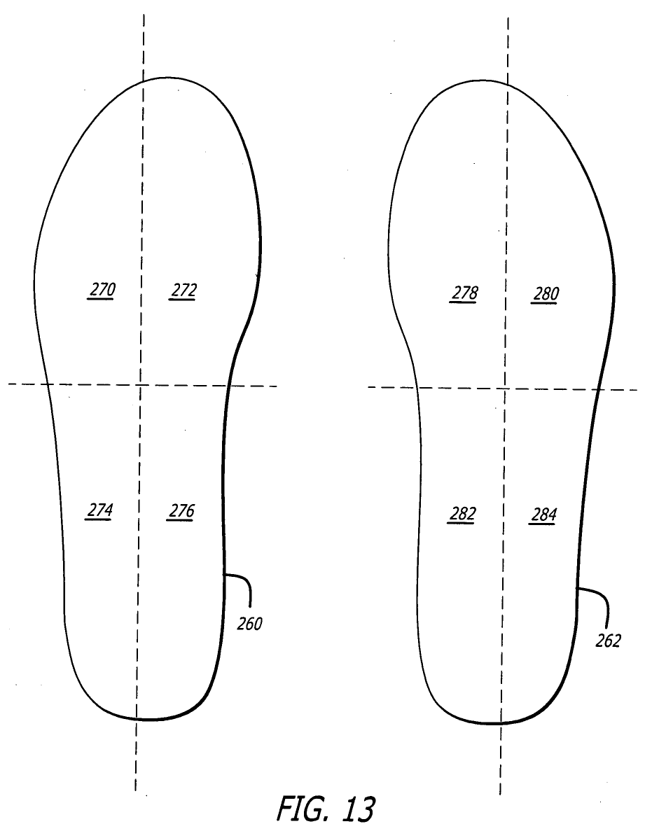

FIG. 13 is a bottom plan view of insole base outlines 260 and 262 of light therapy devices showing broad anatomic zones corresponding to quadrants of a pair of human feet. The predetermined quadrants or anatomic zones of the human foot may include a right front quadrant of the right foot 270, a left front quadrant of the right foot 272, a right rear quadrant of the right foot 274, a left rear quadrant of the right foot 276, a right front quadrant of the left foot 278, a left front quadrant of the left foot 280, a right rear quadrant of the left foot 282 and a left rear quadrant of the left foot 284. Some embodiments of light therapy devices may be manufactured in kits or groupings of embodiments with emitters 21 disposed or concentrated within one or more of the predetermined anatomic zones discussed above. With regard to the anatomic zones corresponding to reflex points or zones of reflex points for acupuncture, optical acupuncture may be carried out for a desired predetermined organ or organ function by applying treatment with a light therapy device having emitters 21 concentrated in the anatomic zone corresponding to the reflex point zone of the predetermined organ. Embodiments having emitters 21 disposed within one or more quadrants of the right and left foot outlines may be used in order to have light therapy devices in stock at care facilities such as hospitals that allow for some site specific delivery without requiring the care facility to stock an impractical number of models or stocking embodiments with emitters 21 disposed over substantially the entire area of the insole base foot outline which would be expensive to produce and use large amounts of battery life.

Some kit embodiments may include eight light therapy devices for a given foot size, e.g., S (small), M (medium), L (large) and XL (extra large), with each one of the eight devices having emitters 21 concentrated in a different one of the eight quadrants 270, 272, 274, 276, 278, 280 and 282. In this way, if all sizes were stocked, a total of 32 models would be able to effectively and efficiently deliver therapeutic light energy to any one of the eight quadrants of a patient's feet of any size.

With regard to the above detailed description, like reference numerals used therein refer to like elements that may have the same or similar dimensions, materials and configurations. While particular forms of embodiments have been illustrated and described, it will be apparent that various modifications can be made without departing from the spirit and scope of the embodiments of the invention. Accordingly, it is not intended that the invention be limited by the forgoing detailed description.

What is claimed is:

1. A light therapy device comprising an insole base having a top surface configured to be disposed adjacent a human patient's foot and having at least one light emitter which is configured to emit light away from the top surface of the insole base and towards at least one predetermined anatomic zone of the human foot.

2. The light therapy device of claim 1 wherein the top surface of the insole base is configured to be disposed adjacent a bottom surface of a patient's right foot.

3. The light therapy device of claim 1 wherein the top surface of the insole base is configured to be disposed adjacent a bottom surface of a patient's left foot.

4. The light therapy device of claim 1 wherein the insole base is configured to have a thickness to allow the top surface of the insole base to be disposed adjacent a bottom surface of a patient's foot while the patient's foot is disposed within pre-existing footwear of the patient.

5. The light therapy device of claim 1 wherein the insole base is integrated into an article of footwear.

6. The light therapy device of claim 1 wherein the at least one emitter is disposed in an anatomic zone corresponding to a reflex point of the human foot.

7. The light therapy device of claim 6 wherein the acupuncture zone of the human foot is selected from the group of acupuncture zones consisting of a heart acupuncture zone, a kidney acupuncture zone, a pancreas acupuncture zone and a liver acupuncture zone.

8. The light therapy device of claim 1 wherein the at least one emitter is disposed in an anatomic zone corresponding to a large artery of the human foot.

9. The light therapy device of claim 1 wherein the at least one emitter is disposed in an anatomic zone corresponding to a predetermined quadrant of the human foot.

10. The light therapy device of claim 9 wherein the predetermined quadrant of the human foot is selected from a group of predetermined quadrants of the human foot consisting of a right front quadrant of the right foot, a left front quadrant of the right foot, a right rear quadrant of the right foot, a left rear quadrant of the right foot, a right front quadrant of the left foot, a left front quadrant of the left foot, a right rear quadrant of the left foot and a left rear quadrant of the left foot.

11. The light therapy device of claim 1 wherein the at least one emitter is configured to emit energy at a wavelength and intensity which is configured to induce production of nitric acid and improve blood circulation in the patient's foot and adjacent areas.

12. The light therapy device of claim 1 wherein the insole base further comprising a battery.

13. The light therapy device of claim 1 wherein the insole base further comprising a recharge and control circuit.

14. A method of treating a human foot of a patient, comprising
providing a light therapy device including an insole base having a top surface configured to be disposed adjacent a human patient's foot and having at least one light emitter which is configured to emit light away from the top surface of the insole base and towards at least one predetermined anatomic zone of the human foot;
disposing the top surface of the insole base adjacent the patient's foot; and
activating at least one emitter of the light therapy device and delivering therapeutic light to at least one predetermined anatomic zone of the patient's foot.

15. The method of treating a human foot of a patient of claim 14 wherein the at least one anatomic zone comprises a reflex point zone for a body organ of the patient and delivering therapeutic light energy comprises performing light acupuncture.

16. A kit for treatment of a human foot comprising a selection of at least a first light therapy device and a second light therapy device for treatment of a human foot, each light therapy device comprising an insole base having a top surface configured to be disposed adjacent a human patient's foot and having at least one light emitter which is configured to emit light away from the top surface of the respective insole base and towards at least one predetermined anatomic zone of the human foot with the first light therapy device configured to emit therapeutic light energy towards a first anatomic zone and the second light therapy device configured to emit therapeutic laser energy towards a second anatomic zone different from the first anatomic zone.

20. A light therapy device, comprising
a shock absorbing insole base having a top surface configured to be disposed adjacent a human patient's foot and having at least one light emitter which is configured to emit light away from the top surface of the insole base and towards at least one predetermined anatomic zone of the human foot; and
an electronic control and power charge circuit configured to supply a patient with a programmed laser therapy regimen to stimulate increased local circulation of the foot to promote healing of foot conditions which is electrically coupled to a battery and a light source of the at least one emitter.

21. The light therapy device of claim 20 wherein the at least one emitter is positioned in the insole base at an anatomic zone corresponding to a known foot reflex point corresponding to an organ affected by diabetes.

22. The light therapy device of claim 2 wherein the at least one emitter is positioned in the insole base at an anatomic zone corresponding to an organ selected from the group consisting of the pancreas, kidneys, spleen, and heart.

23. The light therapy device of claim 20 further comprising an activation switch electrically coupled to the controller charge circuit, and wherein the activation switch is configured to be activated by reaching a predetermined charge level threshold in a battery electrically coupled to the electronic control and power charge circuit.

24. The light therapy device of claim 20 wherein the controller charge circuit is configured to store and generate a predetermined programmed light therapy treatment upon activation.

25. The light therapy device of claim 20 further comprising a plurality of emitters and wherein the controller charge circuit is configured to control the timing and sequential activation of the emitters.

26. The light therapy device of claim 20 wherein the insole base is configured for insertion into an article of footwear.

27. A method of treating peripheral neuropathy comprising:
providing a light therapy device including an insole base having a top surface configured to be disposed adjacent a human patient's foot and having at least one light emitter which is configured to emit light away from the top surface of the insole base and towards at least one predetermined anatomic zone of the human foot;
disposing the top surface of the insole base adjacent the patient's foot; and
activating at least one emitter of the light therapy device and delivering therapeutic light to at least one predetermined anatomic zone of the patient's foot.
